(19) United States
(12) Patent Application Publication
(10) Pub. No.: US 2009/0214507 A1
(43) Pub. Date: Aug. 27, 2009

(54) METHOD OF TREATING PREVENTING, INHIBITING OR REDUCING DAMAGE TO CARDIAC TISSUE

(75) Inventors: Deepak Srivastava, Dallas, TX (US); Ildiko Bock-Marquette, Dallas, TX (US); Ankor Saxena, Dallas, TX (US); Allan L. Goldstein, Washington, DC (US)

Correspondence Address: ROTHWELL, FIGG, ERNST & MANBECK, P.C., 1425 K STREET, N.W., SUITE 800, WASHINGTON, DC, 20005, US

(73) Assignee: RegeneRx Biopharmaceuticals, Inc., Bethesda, MD (US)

(21) Appl. No.: 11/660,376

(22) PCT Filed: Aug. 19, 2005
(86) PCT No.: PCT/US05/29949

§ 371 Date: Sep. 22, 2008

Related U.S. Application Data
(60) Provisional application No. 60/602,884, filed on Aug. 20, 2004.
(60) Provisional application No. 60/625,112, filed on Nov. 5, 2004.

(51) Int. Cl. A61K 38/45; C12Q 1/00; A61K 38/28; A61K 38/16; A61K 38/08

(52) U.S. Cl. 424/94.5; 435/4; 514/3; 514/4; 514/12; 514/16; 514/17

(57) ABSTRACT

A method of treatment for treating, preventing, inhibiting or reducing damage to coronary tissue includes inducing at least one of a physiological function selected from: up-regulation of or increasing ILK activity in the coronary tissue, up-regulation of or increasing Akt activity in the coronary tissue, down-regulation of or reducing cardiomyocyte cell death in the coronary tissue and hibernation of cardiomyocytes in the coronary tissue. An induction agent capable of inducing such physiological function in a subject is administered to a subject in need of such treatment.

### CROSS-REFERENCE TO RELATED APPLICATION

The present application claims benefit of U.S. Provisional Application Ser. No. 60/602,884, filed Aug. 20, 2004, and U.S. Provisional Application Ser. No. 60/625,112, filed Nov. 5, 2004.

### BACKGROUND OF THE INVENTION

1. Field of the Invention

The present invention relates to the field of treating, preventing, inhibiting or reducing damage to cardiac tissue.

2. Description of the Background Art

Heart disease is a leading cause of death in newborns and in adults.

Coronary artery disease results in acute occlusion of cardiac vessels leading to loss of dependent myocardium. Such events are one of the leading causes of death in the Western world. Because the heart is incapable of sufficient muscle regeneration, survivors of myocardial infarctions typically develop chronic heart failure with over ten million cases in the United States alone. While more commonly affecting adults, heart disease in children is the leading non-infectious cause of death in the first year of life and often involves abnormalities in cardiac cell specification, migration or survival.

There are many causes of myocardial and coronary vessel and tissue injuries, including but not limited to myocardial ischemia, clotting, vessel occlusion, infection, developmental defects or abnormalities and other such myocardial events. Myocardial infarction results from blood vessel disease in the heart. It occurs when the blood supply to part of the heart is reduced or stopped (caused by blockage of a coronary artery, as one example). The reduced blood supply causes injuries to the heart muscle cells and may even kill heart muscle cells. The reduction in blood supply to the heart is often caused by narrowing of the epicardial blood vessels due to plaque. These plaques may rupture causing hemorrhage, thrombus formation, fibrin and platelet accumulation and constriction of the blood vessels.

Recent evidence suggests that a population of extracardiac or intracardiac stem cells may contribute to maintenance of the cardiomyocyte population under normal circumstances. Efforts to promote cardiac repair by introduction or recruitment of exogenous stem cells hold promise but typically involve isolation and introduction of autologous or donor progenitor cells. While the stem cell population may maintain a delicate balance between cell death and cell renewal, it is insufficient for myocardial repair after acute coronary occlusion. Introduction of isolated stem cells may improve myocardial function, but this approach has been controversial, and requires isolation of autologous stem cells or use of donor stem cells along with immunosuppression. Efforts to coax pluripotent embryonic stem cells into a cardiomyocyte lineage remain unsuccessful. Technical hurdles of stem cell delivery and differentiation have thus far prevented broad clinical application of cardiac regenerative therapies.

There remains a need in the art for improved methods and compositions for treating, preventing, inhibiting or reducing damage to cardiac tissue.

### SUMMARY OF THE INVENTION

In accordance with one aspect of the present invention, a method of treatment for treating, preventing, inhibiting or reducing damage to coronary tissue comprises inducing at least one of a physiological function selected from: up-regulation of or increasing ILK activity in said coronary tissue, up-regulation of or increasing Akt activity in said coronary tissue, up-regulation of or increasing phosphatidylinositol 3-kinase (PI31K) activity, down-regulation of or reducing cardiomyocyte cell death in said coronary tissue and hibernation of cardiomyocytes in said coronary tissue. This aspect involves administering to a subject in need of such treatment an induction agent capable of inducing at least one said physiological function in said subject.

### DETAILED DESCRIPTION OF THE INVENTION

Without being bound to any specific theory, the present invention provides that damage to coronary tissue can be prevented, treated, inhibited or reduced by inducing one or more physiological functions, which may include regulatory pathways involved in cardiac function or development.

In order to treat, prevent, inhibit or reduce damage to coronary tissue, the physiological functions which may be induced in accordance with the present invention include: up-regulation of or increasing integrin linked kinase (ILK), up-regulation of or increasing protein kinase B (Akt), up-regulation of or increasing phosphatidylinositol 3-kinase (PI3K) activity, down-regulation of or reducing cardiomyocyte cell death and hibernation of cardiomyocytes. In accordance with one embodiment, at least one of these physiological functions is induced by administering an induction agent capable of inducing one or more of the above physiological functions in a subject in need of treatment. The subject may be a mammal, preferably human.

ILK activity can be up-regulated or increased in accordance with the present invention by greater than 10%, 25%, 50% or 100% by addition of the induction agent. Akt activity can be up-regulated or increased by greater than 10%, 25%, 50% or 100% in accordance with the present invention. Cardiomyocyte cell death can be down-regulated or reduced by greater than 10%, 25%, 50% or up to zoo % by utilizing an induction agent in accordance with the present invention. Hibernation of cardiomyocytes can be increased by greater than 10%, 25%, 50% or up to 100% by utilizing an induction in accordance with the present invention. PI3K activity can be up-regulated or increased by greater than 10%, 25%, 50% or 100% by addition of an induction agent in accordance with the present invention.

In accordance with one embodiment, the induction agent is thymosin β4 (Tβ4 or Tβ4). Thymosin β4 was initially identified as a protein that is up-regulated during endothelial cell migration and differentiation in vitro. Thymosin β4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

In accordance with another embodiment, the invention utilizes an induction agent other than Tβ4 for treating, preventing, inhibiting or reducing damage to coronary tissue. Such induction agents may include Tβ4 isoforms, analogues or derivatives, including oxidized Tβ4, Tβ4 sulfoxide, N-terminal variants of Tβ4, C-terminal variants of Tβ4 and antagonists of Tβ4.

Many Tβ4 isoforms have been identified and have about 70%, or about 75%, or about 80% or more homology to the known amino acid sequence of Tβ4. Such isoforms include, for example, Tβ4^{ala}, Tβ9, Tβ10, Tβ11, Tβ12, Tβ13, Tβ14 and Tβ15. These isoforms, along with Tβ4, share an amino acid sequence, LKKTET, that may be involved in treating, preventing, inhibiting or reducing damage to cardiac tissue.

International Application Serial No. PCT/US99/17282, incorporated herein by reference, discloses isoforms of Tβ4 which may be useful in accordance with the present invention as well as amino acid sequence LKKTET and conservative variants thereof, which may be utilized with the present invention. International Application Serial No. PCT/GB99/00833 (WO 99/49883), incorporated herein by reference, discloses oxidized Thymosin β4 which may be utilized in accordance with the present invention.

Thus, it is specifically contemplated that induction agents such as known Tβ4 isoforms, such as those listed above, as well as Tβ4 isoforms not yet identified, will be useful in the methods of the invention.

In addition, other induction agent molecules which may be useful in treating, preventing, inhibiting or reducing damage to cardiac tissue can similarly be employed in the methods of the invention. Such molecules may include gelsolin, vitamin D binding protein (DBP), profilin, cofilin, adsevertin, propomyosin, fincilin, depactin, DnaseI, vilin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, DnaseI, vilin, fragmin, severin, capping protein, β-actinin and acumentin as set forth herein.

Thus, in accordance with one aspect, the present invention may utilize induction agents such as peptides or peptide fragments comprising or consisting essentially of amino acid sequence LKKTET or conservative variants thereof, including amino acid sequences KLKKTET and/or LKKTETQ (collectively sometimes referred to as LKKTET peptides).

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

The invention also is applicable to utilization of induction agents which stimulate production in coronary tissue of one or more of the other herein-described induction agents. Such agents may also be termed “induction initiating agents”. Thus, in accordance with one embodiment, subjects are treated with an agent that stimulates production in the subject of an induction agent as described herein. Thus, an induction agent utilized in accordance with the present invention may directly or indirectly induce at least one physiological function described above so as to treat, prevent, inhibit or reduce damage to coronary tissue. In accordance with one embodiment, induction agents which indirectly induce at least one of the above-described physiological functions so as to treat, prevent, inhibit or reduce damage to coronary tissue may stimulate production of LKKTET peptide, such as Tβ4, in the coronary tissue so as to prevent damage to the coronary tissue.

Without being bound to any particular theory, the phosphatidylinositol 3-kinase (PI3K) and the integrin-linked kinase (ILK) and AkT signaling pathways which may be upregulated by thymosin β4 (Tβ4) or other LKKTET peptides may mediate survival signals and thus play an important role in preventing damage to cardiac tissue after an ischemic insult. AkT is a serine-threonine kinase which may play a role in cell and tissue survival by influencing a number of downstreaming pathways which may inhibit apoptosis. The PI3K and ILK kinases also may activate AkT following stimulation with a variety of membrane receptors, hormones, cytokines, chemokines, and other cellular molecules. Thus, the induction agent utilized in accordance with the present invention may be other than Tβ4 or another LKKTET peptide. Examples of such induction agents may be selected from the following, which is not intended to be limiting: membrane receptors, including the HER (or Erb B) family of growth factor receptors and the estrogen (ER) receptor; insulin or albumin-bound palmitate together with insulin; fibronectin; glutathione; mannitol; inhibitors of p38-MAPK, e.g., SB-203580; erythropoietin; and Rho family proteins such as Ras, Cdc42 and Rac1. Several downstream targets of Akt may include the transcriptional factors BAD and Forkhead, among others. Tβ4 induced Akt activation, as an example, may suppress apoptosis by phosphorylating BAD which then may suppress the release of mitochondrial cytochrome c release and caspase-9 activation. AkT also may activate IKK which may activate nuclear factor-KB (NF-κB) via an inhibitor of NFκB degradation. NFκB then may translocate to the nucleus and induce the transcription of anti-apoptotic genes. Several of the above molecules and other drugs and small molecules may also act synergistically with an induction agent as described herein to inhibit damage to cardiac tissue. Examples of such compounds may be selected from the following, which is not intended to be limiting: aldose reductase inhibitors (ARI) e.g., zopolrestat and others; ACE inhibitors—e.g. ramipril and others; sorbitol dehydrogenase inhibitors e.g. CP-470, 711; M-acetylcysteine (NAC); tyrosine phosphatase inhibitors, e.g., Na orthovanadate; rexinoids (insulin-sensitizing activity of RXR agonists), i.e., class of nuclear receptor ligands having insulin-sensitizing activity, e.g., LG268; salicylates and pharmacological inhibitors of c-Jun N terminal kinase (JNK) and others; clozapine and olanzapine, (atypical antipsychotics); inhibitors of ROS; and inhibitors of BAX.

In one embodiment, the invention provides a method for treating, preventing, inhibiting or reducing coronary damage in a subject by contacting the damaged site with an effective amount of an induction agent as described herein. The contacting may be direct or systemically. Examples of contacting the damaged site include contacting the site with a composition comprising an induction agent as described herein or in combination with at least one agent that enhances penetration of an induction agent as described herein, or delays or slows release of an induction agent as described herein into the area to be treated.

Administration may include, for example, injection directly into cardiac tissue such as heart muscle tissue, intravenous, intraperitoneal, intramuscular or subcutaneous injections, or inhalation, transdermal or oral administration of a composition containing an induction agent as described herein.

An induction agent as described herein may be administered in any suitable coronary tissue damage-treating, -preventing, -inhibiting or -reducing amount. For example, an induction agent as described herein may be administered in dosages within the range of about 0.001-1,000,000 micrograms, more preferably in amounts within the range of about 0.1-5,000 micrograms, most preferably within the range of about 1-30 micrograms.

An induction agent in accordance with the present invention can be administered as a single administration, daily, every other day, etc., for multiple days, weeks or months, etc., with a single administration or multiple administrations per day of administration, such as applications 2, 3, 4 or more times per day of administration.

Tβ4 has been localized to a number of tissue and cell types, and thus agents which stimulate the production of Tβ4, an LKKTET peptide and/or another induction agent as herein described, can be added to or comprise a composition to effect Tβ4 production, LKKTET peptide production and/or production of another induction agent, in cardiac tissue and/or cardiac cells. Such agents may include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF).

Additionally, other agents that assist in treating, preventing, inhibiting or reducing damage to cardiac tissue may be added to a composition along with an induction agent as described herein. Such agents may include angiogenic agents, growth factors, agents that direct differentiation of cells. For example, and not by way of limitation, an induction agent as described herein can be added in combination with any one or more of the following agents: VEGF, KGF, FGF, PDGF, TGFβ, IGF-1, IGF-2, IL-1, prothymosin (c and thymosin α1 in an effective amount.

The invention also includes a pharmaceutical composition comprising a therapeutically effective amount of an induction agent as described herein, in a pharmaceutically acceptable carrier, such as water for injection.

The actual dosage, formulation or composition that treats or prevents damage to cardiac tissue may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable formulations include an induction agent as described herein at a concentration within the range of about 0.001-10% by weight, more preferably within the range of about 0.01-0.1% by weight, most preferably about 0.05% by weight.

The therapeutic approaches described herein involve various routes of administration or delivery of reagents or compositions comprising an induction agent as described herein, including any conventional administration techniques to a subject. The methods and compositions using or containing an induction agent as described herein, and/or other compounds utilized with the invention may be formulated into pharmaceutical compositions by admixture with pharmaceutically acceptable non-toxic excipients or carriers.

The invention may include use of antibodies which interact with an induction agent as described herein, such as an LKKTET peptide or functional fragments thereof. Antibodies which comprise or consist essentially of pooled monoclonal antibodies with different epitopic specificities, as well as distinct monoclonal antibody preparations may be provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of an induction initiating agent which induces gene expression of an induction agent as described herein, such as Tβ4, a Tβ4 isoform or an LKKTET peptide. The term “effective amount” means that amount of agent which effectively induces gene expression of an induction agent as described herein, resulting in effective treatment. An agent which induces gene expression of an induction agent as described herein, may be a polynucleotide. The polynucleotide may be an antisense, a triplex agent, or a ribozyme. For example, an antisense directed to the structural gene region or to the promoter region of Tβ4, a Tβ4 isoform or an LKKTET peptide may be utilized.

In another embodiment, the invention provides a method for utilizing compounds that induce peptide activity of an induction agent as described herein. Compounds that affect activity of an induction agent as described herein (e.g., antagonists and agonists) may include peptides, peptidomimetics, polypeptides, chemical compounds, minerals such as zincs, and biological agents.

The invention further relates to a method of screening for a compound capable of preventing damage to a coronary tissue as described above, comprising contacting a coronary tissue with a candidate compound; and measuring a level of at least one said physiological function in said coronary tissue, wherein an increase of said level of at least one said physiological function compared to a level of at least one said physiological function in a coronary tissue lacking said candidate compound indicates that said compound is capable of treating, preventing, inhibiting or reducing damage to said coronary tissue.

The invention also relates to a method of screening for a compound capable of inducing at least one said physiological function as described above, comprising contacting a coronary tissue with a candidate compound; and measuring Tβ4 activity in said tissue, wherein an increase of Tβ4 activity in said coronary tissue, compared to a level of Tβ4 activity in a coronary tissue lacking said candidate compound, indicates that said compound is capable of inducing at least one said physiological function.

The invention is further illustrated by the following examples, which are not to be construed as limiting.

### Example 1

Synthetic Tβ4 and an antibody to Tβ4 was provided by RegeneRx Biopharmaceuticals, Inc. (3 Bethesda Metro Center, Suite 700, Bethesda, Md. 20814) and were tested in a collagen gel assay to determine their effects on the Transformation of cardiac endothelial cells to mesenchymal cells. It is well established that development of heart valves and other cardiac tissue are formed by epithelial-mesenchymal transformation and that defects in this process can cause serious cardiovascular malformation and injury during development and throughout life. At physiological concentrations Tβ4 markedly enhances the transformation of endocardial cells to mesenchymal cells in the collagen gel assay. Furthermore, an antibody to Tβ4 inhibited and blocked this transformation. Transformation of atrioventricular endocardium into invasive mesenchyme is an aspect of the formation and maintenance of normal cardiac tissue and in the formation of heart valves.

### Example 2

Regulatory pathways involved in cardiac development may have utility in reprogramming cardiomyocytes to aid in cardiac repair. In studies of genes expressed during cardiac morphogenesis, it was found that the forty-three amino acid peptide thymosin β4 was expressed in the developing heart. Thymosin β4 has numerous functions with the most prominent involving sequestration of G-actin monomers and subsequent effects on actin-cytoskeletal organization necessary for cell motility, organogenesis and other cell biological events. Recent domain analyses indicate that β4-thymosins can affect actin assembly based on their carboxy-terminal affinity for actin. In addition to cell motility, thymosin β4 may affect transcriptional events by influencing Rho-dependent gene expression or chromatin remodeling events regulated by nuclear actin.

Here, it is shown that thymosin β4 can stimulate migration of cardiomyocytes and endothelial cells and promote survival of cardiomyocytes. The LIM domain protein PINCH and Integrin Linked Kinase (ILK), both of which are necessary for cell migration and survival, formed a complex with thymosin β4 that resulted in phosphorylation of the survival kinase Akt/PKB. Inhibition of Akt phosphorylation reversed thymosin β4's effects on cardiac cells. Treatment of adult mice with thymosin β4 after coronary ligation resulted in increased phosphorylation of Akt in the heart, enhanced early myocyte survival within twenty-four hours and improved cardiac function. These results indicate that an endogenous protein expressed during cardiogenesis may be re-deployed to protect myocardium in the setting of acute coronary events.

### Results

### Developmental Expression of Thymosin β4

Expression of thymosin β4 in the developing brain was previously reported, as was expression in the cardiovascular system, although not in significant detail. Whole mount RNA in situ hybridization of embryonic day (E) 10.5 mouse embryos revealed thymosin β4 expression in the left ventricle, outer curvature of the right ventricle and cardiac outflow tract. Radioactive in situ hybridization indicated that thymosin β4 transcripts were enriched in the region of cardiac valve precursors known as endocardial cushions. Cells in this region are derived from endothelial cells that undergo mesenchymal transformation, migrate away from the endocardium and invade a swelling of extracellular matrix separating the myocardium and endocardium. In addition to endocardial cells, a subset of myocardial cells migrate and populate the cushion region and this process is necessary for separation and remodeling of the cardiac chambers. Using immunohistochemistry, it was found that thymosin β4-expressing cells in the cushions also expressed cardiac muscle actin, suggesting that thymosin β4 was present in migratory cardiomyocytes that invade the endocardial cushion. Finally, thymosin β4 transcripts and protein were also expressed at E9.5-E11.5 in the ventricular septum and the less differentiated, more proliferative region of the myocardium, known as the compact layer, which migrates into the trabecular region as the cells mature. Outflow tract myocardium that migrates from the anterior heart field also expressed high levels of thymosin β4 protein.

### Secreted Thymosin β4 Stimulates Cardiac Cell Migration and Survival

Although thymosin β4 is found in the cytosol and nucleus and functions intracellularly, we found that conditioned medium of Cos 1 cells transfected with myc-tagged thymosin β4 contained thymosin β4 detectable by Western blot, consistent with previous reports of thymosin β4 secretion and presence in wound fluid. Upon expression of thymosin β4 on the surface of phage particles added extracellularly to embryonic cardiac explants, it was found that an anti-phage antibody coated the cell surface and was ultimately detected intracellularly in the cytosol and nucleus while control phage was not detectable. Similar observations were made using biotinylated thymosin β4. These data indicated that secreted thymosin β4 may be internalized into cells, although the mechanism of cellular entry remains to be determined.

To test the effects of secreted thymosin β4 on cardiac cell migration, an embryonic heart explant system designed to assay cell migration and transformation events on a three-dimensional collagen gel was utilized. In this assay, explants of adjacent embryonic myocardium and endocardium from valve-forming regions were placed on a collagen gel with the endocardium adjacent to the collagen. Signals from cardiomyocytes induce endocardial cell migration but myocardial cells do not normally migrate onto the collagen in significant numbers. In contrast, upon addition of thymosin β4 to the primary explants, it was observed that a large number of spontaneously beating, cardiac muscle actin-positive cells had migrated away from the explant. No significant difference in cell death or proliferative rate based on TUNEL assay or phosho-histone H3 immunostaining, respectively, was observed in these cells compared to control cells.

To test the response of post-natal cardiomyocytes, primary rat neonatal cardiomyocytes were cultured on laminin-coated glass and treated the cells with phosphate buffered saline (PBS) or thymosin β4. Similar to embryonic cardiomyocytes, it was found that the migrational distance of thymosin β4-treated neonatal cardiomyocytes was significantly increased compared to control (p<0.05). In addition to thymosin β4's effects on myocardial cell migration, a similar effect was observed on endothelial migration in the embryonic heart explant assay. Exposure of E11.5 explants to thymosin β4 resulted in an increased number of migrating endothelial cells, compared to PBS (p<0.01).

Primary culture of neonatal cardiomyocytes typically survived for approximately one to two weeks with some cells beating up to two weeks when grown on laminin-coated slides in our laboratory. Surprisingly, neonatal cardiomyocytes survived significantly longer upon exposure to thymosin β4 with rhythmically contracting myocytes visible for up to 28 days. In addition, the rate of beating was consistently faster in thymosin β4-treated neonatal cardiomyocytes (95 vs. 50 beats per minute, p<0.02), indicating either a change in cell-cell communication or more vigorous cardiomyocytes.

### Thymosin β4 Activates ILK and Akt/Protein Kinase B

To investigate the potential mechanisms through which thymosin β4 might be influencing cell migration and survival events, thymosin β4 interacting proteins were searched. The amino-terminus of thymosin β4 was fused with affi-gel beads resulting in exposure of the carboxy-terminus that allowed identification of previously unknown interacting proteins but prohibited association with actin. An E9.5-12.5 mouse heart T7 phage cDNA library was synthesized and screened by phage display and thymosin β4-interacting clones were enriched and confirmed by ELISA. PINCH, a LIM domain protein, was most consistently isolated in this screen and interacted with thymosin β4 in the absence of actin (ELISA). PINCH and integrin linked kinase (ILK) interact directly with one another and indirectly with the actin cytoskeleton as part of a larger complex involved in cell-extracellular matrix interactions known as the focal adhesion complex. PINCH and ILK are required for cell motility and for cell survival, in part by promoting phosphorylation of the serine-threonine kinase Akt/protein kinase B, a central kinase in survival and growth signaling pathways. Plasmids encoding thymosin β4 were transfected with or without PINCH or ILK in cultured cells and it was found that thymosin β4 co-precipitated with PINCH or ILK independently. Moreover, PINCH, ILK and thymosin β4 consistently immunoprecipitated in a common complex, although the interaction of ILK with thymosin β4 was weaker than with PINCH. The PINCH interaction with thymosin β4 mapped to the fourth and fifth LIM domains of PINCH while the amino terminal ankryin domain of ILK was sufficient for thymosin β4 interaction.

Because recruitment of ILK to the focal adhesion complex is important for its activation, the effects of thymosin β4 on ILK localization and expression were assayed. ILK detection by immunocytochemistry was markedly enhanced around the cell edges after treatment of embryonic heart explants or C2C12 myoblasts with synthetic thymosin β4 protein (10 ng/100 ul) or thymosin β4-expressing plasmid. Western analysis indicated a modest increase in ILK protein levels in C2C12 cells, suggesting that the enhanced immunofluorescence may be in part due to altered localization by thymosin β4. It was found that upon thymosin β4 treatment of C2C 12 cells, ILK was functionally activated, evidenced by increased phosphorylation of its known substrate Akt, using a phospho-specific antibody to serine 473 of Akt, while total Akt protein was unchanged. The similar effects of extracellularly administered thymosin β4 and transfected thymosin β4 were consistent with previous observations of internalization of the peptide and suggested an intracellular rather than an extracellular role in signaling for thymosin β4. Because thymosin β4 sequesters the pool of G-actin monomers, the effects on ILK activation were dependent on thymosin β4's role in regulating the balance between polymerized F-actin and monomeric G-actin were tested. F-actin polymerization was inhibited using C3 transferase and also F-actin formation was promoted with an activated Rho, but neither intervention affected the ILK activation observed after treatment of COS1 or C2C 12 cells with thymosin β4.

To determine if activation of ILK was necessary for the observed effects of thymosin β4, a well-described ILK inhibitor, wortmannin, was employed, which inhibits ILK's upstream kinase, phosphatidylinositol 3-kinase (PI3-kinase). Using myocardial cell migration and beating frequency as assays for thymosin β4 activity, embryonic heart explants were cultured as described above in the presence of thymosin β4 with or without wortmannin. Consistent with ILK mediating thymosin β4's effects, a significant reduction in myocardial cell migration and beating frequency was observed upon inhibition of ILK (p<0.05). Together, these results supported a physiologically significant interaction of thymosin β4-PINCH-ILK within the cell and suggested that this complex may mediate some of the observed effects of thymosin β4 relatively independent of actin polymerization.

Thymosin β4 Promotes Cell Survival after Myocardial Infarction and Improves Cardiac Function

Because of thymosin β4's effects on survival and migration of cardiomyocytes cultured in vitro and phosphorylation of Akt, it was tested whether thymosin β4 might aid in cardiac repair in vivo after myocardial damage. Myocardial infarctions in fifty-eight adult mice were created by coronary artery ligation and treated half with systemic, intracardiac, or systemic plus intracardiac thymosin β4 immediately after ligation and the other half with PBS. Intracardiac injections were done with collagen (control) or collagen mixed with thymosin β4. All forty-five mice that survived two weeks later were interrogated for cardiac function by random-blind ultrasonography at 2 and 4 weeks after infarction by multiple measurements of cardiac contraction. Four weeks after infarction, left ventricles of control mice had a mean fractional shortening of 23.2+/−1.2% (n=22, 95% confidence interval); in contrast, mice treated with thymosin β4 had a mean fractional shortening of 37.2+/−1.8% (n=23, 95% confidence intervals; p<0.001). As a second measure of ventricular function, two-dimensional echocardiographic measurements revealed that the mean fraction of blood ejected from the left ventricle (ejection fraction) in thymosin β4 treated mice was 57.7+/−3.2% (n=23, 95% confidence interval; p<0.0001) compared to a mean of 28.2+/−2.5% (n=22, 95% confidence interval) in control mice after coronary ligation. The greater than 60% or 100% improvement in cardiac fractional shortening or ejection fraction, respectively, suggested a significant improvement with exposure to thymosin β4, although cardiac function remained depressed compared to sham operated animals (˜60% fractional shortening; ˜75% ejection fraction). Finally, the end diastolic dimensions (EDD) and end systolic dimensions (ESD) were significantly higher in the control group, indicating that thymosin β4 treatment resulted in decreased cardiac dilation after infarction, consistent with improved function. Remarkably, the degree of improvement when thymosin β4 was administered systemically through intraperitoneal injections or only locally within the cardiac infarct was not statistically different, suggesting that the beneficial effects of thymosin β4 likely occurred through a direct effect on cardiac cells rather than through an extracardiac source. Control cardiac injections were performed with the same collagen vehicle making it unlikely that an endogenous reaction to the injection contributed to the cardiac recovery.

To determine the manner in which thymosin β34 improved cardiac function, multiple serial histologic sections of hearts treated with or without thymosin β4 were examined. Trichrome stain at three levels of section revealed that the size of scar was reduced in all mice treated with thymosin β4 but was not different between systemic or local delivery of thymosin β4, consistent with the echocardiographic data above. Quantification of scar volume using six levels of sections through the left ventricle of a subset of mice demonstrated significant reduction of scar volume in thymosin β4 treated mice (p<0.05). We did not detect significant cardiomyocyte proliferation or death at three, six, eleven or fourteen days after coronary ligation in PBS or thymosin β4 treated hearts. However, twenty-four hours after ligation we found a striking decrease in cell death by TUNEL assay (green) in thymosin β4 treated cardiomyocytes, confirmed by double-labeling with muscle-actin antibody (red). TUNEL positive cells that were also myocytes were rare in the thymosin β4 group but abundant in the control hearts. Consistent with this observation, it was found that the left ventricle fractional shortening three days after infarction was 39.2+/−2.34% (n=4, 95% confidence interval) with intracardiac thymosin β4 treatment compared to 28.8+/−2.26% (n=4, 95% confidence interval) in controls (p<0.02); ejection fraction was 64.2+/−6.69% or 44.7+/−8.4%, respectively (p<0.02), suggesting early protection by thymosin β4. Finally, there was no detection of any differences in the number of c-kit, Sca-1 or Abcg2 positive cardiomyocytes between treated and untreated hearts and the cell volume of cardiomyocytes in thymosin β4 treated animals was similar to mature myocytes, suggesting that the thymosin β4-induced improvement was unlikely to be influenced by recruitment of known stem cells into the cardiac lineage. Thus, the decreased scar volume and preserved function of thymosin β4 treated mice were likely due to early preservation of myocardium after infarction through thymosin β4's effects on survival of cardiomyocytes.

Because thymosin β4 upregulates ILK activity and Akt phosphorylation in cultured cells, the effects on these kinases in vivo were tested. By western blot it was found that the level of ILK protein was increased in heart lysates of mice treated with thymosin β4 after coronary ligation compared with PBS treated mice. Correspondingly, phospho-specific antibodies to Akt-5473 revealed an elevation in the amount of phosphorylated Akt-5473 in mice treated with thymosin β4, consistent with the effects of thymosin β4 on ILK described earlier. Total Akt protein was not increased. These observations in vivo were consistent with the effects of thymosin β4 on cell migration and survival demonstrated in vitro and suggest that activation of ILK and subsequent stimulation of Akt may in part explain the enhanced cardiomyocyte survival induced by thymosin β4, although it is unlikely that a single mechanism is responsible for the full repertoire of thymosin β4's cellular effects.

### DISCUSSION

The evidence presented here suggests that thymosin β4, a protein involved in cell migration and survival during cardiac morphogenesis, may be re-deployed to minimize cardiomyocyte loss after cardiac infarction. Given the roles of PINCH, ILK and Akt, the data is consistent with this complex playing a central role in thymosin β4's effects on cell motility, survival and cardiac repair. Thymosin β4's ability to prevent cell death within twenty four hours after coronary ligation likely leads to the decreased scar volume and improved ventricular function observed in mice. Although thymosin β4 activation of ILK is likely to have many cellular effects, the activation of Akt may be the dominant mechanism through which thymosin β4 promotes cell survival. This is consistent with Akt's proposed effect on cardiac repair when over-expressed in mouse marrow-derived stem cells administered after cardiac injury, although this likely occurs in a non-cell autonomous fashion.

The early effect of thymosin β4 in protecting the heart from cell death was reminiscent of myocytes that are able to survive hypoxic insult by “hibernating”. While the mechanisms underlying hibernating myocardium are unclear, alterations in metabolism and energy usage appear to promote survival of cells. Induction agents such as thymosin β4 may alter cellular properties in a manner similar to hibernating myocardium, possibly allowing time for endothelial cell migration and new blood vessel formation.

Here, we show that the G-actin sequestering peptide thymosin β4 promotes myocardial and endothelial cell migration in the embryonic heart and retains this property in post-natal cardiomyocytes. Survival of embryonic and postnatal cardiomyocytes in culture was also enhanced by thymosin β4. It was found that thymosin β4 formed a functional complex with PINCH and Integrin Linked Kinase (ILK), resulting in activation of the survival kinase Akt/PKB, which was necessary for thymosin β4's effects on cardiomyocytes. After coronary artery ligation in mice, thymosin β4 treatment resulted in upregulation of ILK and Akt activity in the heart, enhanced early myocyte survival and improved cardiac function. These findings indicate that thymosin β4 promotes cardiomyocyte migration, survival and repair and is a novel therapeutic target in the setting of acute myocardial damage.

### Methods

### RNA In Situ Hybridization

Whole-mount or section RNA in situ hybridization of E 9.5-12.5 mouse embryos was performed with digoxigenin-labeled or S-labelled antisense riboprobes synthesized from the 3′ UTR region of mouse thymosin β4 cDNA that did not share homology with the closely related transcript of thymosin β10.

### Immunohistochemistry

Embryonic or adult cardiac tissue was embedded in paraffin and sections used for immunohistochemistry. Embryonic heart sections were incubated with anti-thymosin β4 that does not recognize thymosin β10. Adult hearts were sectioned at ten equivalent levels from the base of the heart to the apex. Serial sections were used for trichrome sections and reaction with sarcomeric a-actinin, c-kit, Sca-1, Abcg2, and BrdU antibodies and for TUNEL assay (Intergen Company #S7111).

### Collagen Gel Migration Assay

Outflow tract was dissected from E11.5 wild type mouse embryos and placed on collagen matrices as previously described. After 10 hours of attachment explants were incubated in 30 ng/300 μl thymosin β4 in PBS, PBS alone or thymosin β4 and 100 nM wortmannin. Cultures were carried out for 3-9 days at 37° C. 5% CO₂ and fixed in 4% paraformaldehyde in PBS for 10 min at RT. Cells were counted for quantification of migration and distance using at least three separate explants under each condition for endothelial migration and eight separate explants for myocardial migration.

### Immunocytochemistry on Collagen Gel Explants

Paraformaldehyde-fixed explants were permeabilized for 10 min at RT with Permeabilize solution (10 mM PIPES pH6.8; 50 mMNaC1; 0.5% Triton X-100; 300 mM Sucrose; 3 mM MgC1₂) and rinsed with PBS 2×5 min at RT. After a series of blocking and rinsing steps, detection antibodies were used and explants rinsed and incubated with Equilibration buffer (Anti-Fade kit) 10 min at room temperature. Explants were scooped to a glass microscope slide, covered, and examined by fluorescein microscopy. TUNEL assay was performed using ApopTag Plus Fluorescein In Situ Apoptosis detection kit (Intergen Company #S7111) as recommended.

Embryonic T7 Phage Display cDNA Library

Equal amounts of mRNA were isolated and purified from E 9.5-12.5 mouse embryonic hearts by using Straight A's mRNA Isolation System (Novagen, Madison Wis.). cDNA was synthesized by using T7Selectlo-3 OrientExpress cDNA Random Primer Cloning System (Novagen, Madison Wis.). The vector T7Selectlo-3 was employed to display random primed cDNA at the C-terminus of 5-15 phage 10B coat protein molecules. Expression of the second coat protein 10A was induced. After EcoRl and Hind III digestion, inserts were ligated into T7 selectlo-3 vector (T7 select System Manual, Novagen). The vector was packaged and complexity of the library was 10⁷. Packaged phage was amplified in a log phase 0.5 L culture of BLT5615 E. Coli strain at 37° C. for 4 h. The cell debris was removed by centrifugation and the phage was precipitated with 8% polyethylene glycol. Phage was extracted from the pellet with 1M NaCl/10 mM Tris-HCl pH 8.0/1 mM EDTA and purified by CsCI gradient ultracentrifugation. Purified phages were dialyzed against PBS and stored in 10% glycerol at −80° C.

### T7 Phage Biopanning

300 ul of Affi-Gel 15 (Bio-Rad Laboratories) was coupled with 12 ug of synthesized thymosin β4 protein (RegeneRx) following the manufacturers manual, likely via amino terminal lysine residues. After blocking with 3% BSA in PBS for 1 h the gel was transferred to a column and washed with 10 ml of PBS, 2 ml of 1% SDS/PBS and 1 ml of PBS/0.05% Tween-20 (PBST)×4. 10⁹ pfu's of the T7 phage embryonic heart library (100× of the complexity) in 500 ul of PBST was applied to the column and incubated for 5 min to achieve low stringency biopanning. Unbound phages were washed with 50 m1 of PBS. Bound phages were eluted in 2.0 ml of 1% SDS. 10 μl of eluted phages was titered and the rest of the phages were immediately amplified in 0.5 L of log phase BLT5615 E. Coli culture until lysis. Cell debris was removed by centrifugation, lysate was titered and 10⁹ pfu's of phages were used for the next round of biopanning. 4 rounds of biopanning were performed and 30 single colonies were picked after the 2^{nd} 3^{rd} and 4^{th} round before amplification, respectively for sequence analysis. Single colonies containing greater than ten amino acids were amplified and used for ELISA confirmation assay.

### ELISA Confirmation Assay

MaxiSorp Nunc-Immuno Plates (Nalgene Nunc International) were coated with 1 μg/100 μl of synthesized thymosin β4 peptide overnight then washed with PBS and blocked with 3% BSA. 10⁹ pfu's of amplified single phage colonies were added in PBST to each well separately and incubated for 1.5 h at RT. T7 wild type phage was used as negative control. Unbound phages were removed by washing with PBS (×4), and bound phages were eluted by adding 200 μl of 1% SDS/PBS to the wells for 1 h at RT.

### Coimmunoprecipitation

Cos and 10 T1/2 cells were transfected with thymosin β4, PINCH and/or ILK and lysates precipitated with antibodies to each as previously described. Western blots were performed using anti-ILK polyclonal antibody (Santa Cruz), anti-thymosin β4 polyclonal antibody and anti-myc or anti-FLAG antibody against tagged versions of PINCH.

### Animals and Surgical Procedures

Myocardial infarction was produced in fifty-eight male C57BL/6J mice at 16 weeks of age (25-30 g) by ligation of the left anterior descending coronary artery as previously described. Twenty-nine of the ligated mice received thymosin β4 treatment immediately following ligation and the remaining twenty-nine received PBS injections. Treatment was given intracardiac with thymosin β4 (200 ng in 10 ul collagen) or with 10 ul of collagen; intraperitoneally with thymosin β4 (150 μg in 300 μl PBS) or with 3000 of PBS; or by both intracardiac and intraperitoneal injections. Intraperitoneal injections were given every three days until mice were sacrificed. Doses were based on previous studies of thymosin β4 biodistribution. Hearts were removed, weighed and fixed for histologic sectioning. Additional mice were operated on in a similar fashion for studies 0.5, 1, 3, 6 and 11 days after ligation.

### Analysis of Cardiac Function by Echocardiography

Echocardiograms to assess systolic function were performed using M-mode and 2-dimensional measurements as described previously. The measurements represented the average of six selected cardiac cycles from at least two separate scans performed in random-blind fashion with papillary muscles used as a point of reference for consistency in level of scan. End diastole was defined as the maximal left ventricle (LV) diastolic dimension and end systole was defined as the peak of posterior wall motion. Single outliers in each group were omitted for statistical analysis. Fractional shortening (FS), a surrogate of systolic function, was calculated from LV dimensions as follows: FS=EDD−ESD/EDD×100%. Ejection fraction (EF) was calculated from two-dimensional images. EDD, end diastolic dimension; ESD, end systolic dimension.

### Calculation of Scar Volume

Scar volume was calculated using six sections through the heart of each mouse using Openlab 3.03 software (Improvision) similar to previously described. Percent area of collagen deposition was measured on each section in blinded fashion and averaged for each mouse.

### Statistical Analyses

Statistical calculations were performed using standard t-test of variables with 95% confidence intervals.

Thymosin β4 promotes myocardial and endothelial cell migration in the embryonic heart and retains this property in postnatal cardiomyocytes. Survival or embryonic and postnatal cardiomyocytes in culture was also enhanced by thymosin β4. Thymosin β4 forms a functional complex with PINCH and integrin-linked kinase (ILK), resulting in activation of the survival kinase Akt (also know as protein kinase B). After coronary artery ligation in mice, thymosin β4 treatment results in upregulation of ILK and Akt activity in the heart, enhances early myocyte survival and improves cardiac function. These findings indicate that thymosin β4 promotes cardiomyocyte migration, survival and repair and the pathway it regulates is a new therapeutic target in the setting of acute myocardial damage.

1. A method of treatment for treating, preventing, inhibiting or reducing damage to coronary tissue comprising inducing at least one of a physiological function selected from: up-regulation of or increasing ILK activity in said coronary tissue, up-regulation of or increasing Akt activity in said coronary tissue, up-regulation of or increasing phosphatidylinositol 3-kinase (PI3K) activity in said coronary tissue, down-regulation of or reducing cardiomyocyte cell death in said coronary tissue and hibernation of cardiomyocytes in said coronary tissue, by administering to a subject in need of said treatment an induction agent capable of inducing said physiological function in said subject.

2. The method of claim 1 wherein said induction agent is thymosin beta 4 (Tβ4), a Tβ4 isoform, analogue or derivative, oxidized Tβ4, an N-terminal variant of Tβ4, a C-terminal variant of Tβ4, an agonist of Tβ4, Tβ4^{ala}, Tβ9, Tβ10, Tβ11, Tβ12, Tβ13, Tβ14, Tβ15, gelsolin, vitamin D binding protein (DBP), profilin, cofilin, adsevertin, propomyosin, fincilin, depactin, Dnasel, vilin, fragmin, severin, capping protein, β-actinin or acumentin.

3. The method of claim 1 wherein said induction agent is an LKKTET peptide.

4. The method of claim 3 wherein said induction agent is Tβ4.

5. The method of claim 1 wherein said induction agent is other than Tβ4.

6. The method of claim 5 wherein said polypeptide comprises amino acid sequence LKKTET [SEQ ID NO:1], amino acid sequence KLKKTET [SEQ ID NO:2], amino acid sequence LKKTETQ [SEQ ID NO:3], and N-terminal variant of Tβ4, a C-terminal variant of Tβ4, an isoform of Tβ4, oxidized Tβ4 or Tβ4 sulfoxide.

7. The method of claim 1 wherein said induction agent directly or indirectly induces said physiological function.

8. The method of claim 6 wherein said induction agent indirectly induces said physiological function, and said induction agent stimulates production of an LKKTET peptide in said coronary tissue.

9. The method of claim 7 wherein said induction agent is other than an LKKTET peptide.

10. The method of claim 9 wherein said induction agent is selected from at least one of membrane receptors, HER growth factor receptors, Erb B growth factor receptors, estrogen (ER) receptor, insulin, albumin-bound palmitate in combination with insulin, fibronectin, glutathione, mannitol, inhibitors of p38-MAPK, SB-203580, erythropoietin, Rho family proteins, Ras, Cdc42 or Rac1.

11. The method of claim 10 further comprising administering to said subject an effective amount of at least one molecule selected from aldose reductase inhibitors (ARI), zopolrestat, ACE inhibitors, ramipril, sorbitol dehydrogenase, inhibitors, CP-470, CP-711; M-acetylcysteine (NAC), tyrosine phosphatase inhibitors, e.g., Na orthovanadate, rexinoid nuclear receptor ligands having insulin-sensitizing activity, salicylates, pharmacological inhibitors of c-Jun N terminal kinase (JNK), clozapine, olanzapine, inhibitors of ROS, or inhibitors of BAX.

12. The method of claim 1 further comprising administering to said subject an effective amount of a molecule selected from aldose reductase inhibitors (ARI), zopolrestat, ACE inhibitors, ramipril, sorbitol dehydrogenase, inhibitors, CP-470, CP-711; M-acetylcysteine (NAC), tyrosine phosphatase inhibitors, e.g., Na orthovanadate, rexinoid nuclear receptor ligands having insulin-sensitizing activity, salicylates, pharmacological inhibitors of c-Jun N terminal kinase (JNK), clozapine, olanzapine, inhibitors of ROS, or inhibitors of BAX.

13. The method of claim 1 wherein said physiological function comprises up-regulation of or increasing ILK activity in said coronary tissue.

14. The method of claim 1 wherein said physiological function comprises up-regulation of or increasing Akt activity in said coronary tissue.

15. The method of claim 1 wherein said physiological function comprises up-regulation of or increasing phosphatidylinositol 3-kinase (PI3K) activity in said coronary tissue.

16. The method of claim 1 wherein said physiological function comprises down-regulation of or reducing cardiomyocyte cell death in said coronary tissue.

17. The method of claim 1 wherein said physiological function comprises hibernation of cardiomyocytes in said coronary tissue.

18. The method of claim 1 wherein said induction agent is administered to said subject at a dosage within a range of about 0.001-1,000,000 micrograms.

19. The method of claim 1 wherein said induction agent is administered by direct injection into said coronary tissue, intravenous, intraperitoneal, intramuscular, subcutaneous, inhalation, transdermal or oral administration.

20. The method of claim 1 wherein said induction agent is administered to said subject at a dosage within a range of about 0.1-5,000 micrograms.

21. The method of claim 1 wherein said induction agent is administered to said subject at a dosage within a range of about 1-30 micrograms.

22. The method of claim 21 wherein said induction agent is Tβ4.

23. The method of claim 8 wherein said LKKTET peptide is Tβ4.

24. A method of screening for a compound capable of preventing damage to a coronary tissue in accordance with the method of claim 1, comprising contacting a coronary tissue with a candidate compound; and measuring a level of at least one said physiological function in said coronary tissue, wherein an increase of said level of at least one said physiological function compared to a level of at least one said physiological function in a coronary tissue lacking said candidate compound indicates that said compound is capable of treating, preventing, inhibiting or reducing damage to said coronary tissue.

25. The method of claim 24 wherein said compound is an LKKTET peptide other than Tβ4.

26. A method of screening for a compound capable of inducing at least one said physiological function in accordance with the method of claim 1, comprising contacting a coronary tissue with a candidate compound; and measuring Tβ4 activity in said tissue, wherein an increase of Tβ4 activity in said contact of coronary tissue, compared to a level of Tβ4 activity in a coronary tissue lacking said candidate compound, indicates that said compound is capable of inducing at least one said physiological function.


SEQUENCE LISTING (3 sequences)

SEQ ID NO: 1 - protein, 6 aa, Homo sapiens
LKKTET

SEQ ID NO: 2 - protein, 7 aa, Homo sapiens
KLKKTET

SEQ ID NO: 3 - protein, 7 aa, Homo sapiens
LKKTETQ